(19) United States
(12) Patent Application Publication
(10) Pub. No.: US 2022/0071621 A1
(43) Pub. Date: Mar. 10, 2022

(54) TAPERED TRANSITION PORTION FOR SUTURE NEEDLES

(71) Applicant: ErgoSurgical Group Corp., Santa Maria, PR (US)

(72) Inventor: Luis Jose Almodovar, Santa Maria, PR (US)

(21) Appl. No.: 17/471,017

(22) Filed: Sep. 9, 2021

Related U.S. Application Data
(60) Provisional application No. 63/076,925, filed on Sep. 10, 2020.

(51) Int. Cl. A61B 17/062; A61B 17/06

(52) U.S. Cl. CPC A61B 17/062; A61B 17/06066; A61B 17/06166; A61B 2017/06076; A61B 2017/06176; A61B 2017/0608; A61B 2017/06052

(57) ABSTRACT

Disclosed is a suturing needle having a tapered transition portion extending from a proximal end of the needle to cover at least a portion of a suture thread swaged on the needle. The tapered transition portion is flexible and provides a smooth transition from a diameter of the suture thread to a diameter of the needle body. The tapered transition portion allows for engaging the suture needle on rollers of a rotational needle driver in one smooth motion.

### CROSS REFERENCE TO RELATED APPLICATIONS

The present application claims the benefit of U.S. Provisional Application 63/076,925 filed 10 Sep. 2020; which is incorporated herein by reference in its entirety.

### TECHNICAL FIELD

The present disclosure generally relates to suture needles having a tapered transition portion.

### BACKGROUND

Suturing procedures used to close wounds or incisions consume a considerable amount of time during a surgical procedure. Generally, the instruments used in suturing procedures are the suturing material, the suturing needle, and the suturing driver. Efforts made to reduce the time and enhance the safety of the procedure have mainly focused on modifications to the needle driver.

The traditional way of driving a needle has been to clamp it down between two flat tips of a needle driver. This forms a static relationship between the needle and needle driver because the needle, once clamped down between the flat tips, cannot move relative to the driver. Moreover, needle drivers are generally designed to fit right-handed users. Thus, left-handed users have difficulties performing the suturing procedure. The right handedness of those devices further affects the capacity of left-handed surgeons to lock and unlock the drivers' locking mechanism. This increases the risks of negative outcomes for patients from wrong needle driver maneuverings.

Recent advances in needle drivers address these deficiencies, including at least this static relationship of the needle and the handedness of the prior art drivers.

Current suture needles have not been designed for, and therefore cannot optimally perform, this task because they were not designed to work with new roller needle drivers. When trying to drive standard swaged needles with these new ergonomic rotational action needle drivers, the rollers generally need to be opened to move over the swage end of the needle. That is, the swage represents a step or lip on the needle that may be damaged by the rollers, or conversely, that may damage the rollers.

Accordingly, new needle designs that may enable smooth loading of a swaged needle by these new needle drivers are desirable and an object of the present disclosure.

### SUMMARY

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents form part of the common general knowledge in the art.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

The present disclosure provides a suturing needle comprising a needle tip portion having a needle tip at a distal end for piercing tissue; a needle body having a distal end connected to a proximal end of the needle tip portion; a suture thread attached to a proximal end of the needle body by a thread attachment component; and a tapered transition portion, for example, in the form of a ramp, extending from the proximal end of the needle body and covering the thread attachment component and at least a portion of the suture thread.

According to certain embodiments, a first end of the tapered transition portion proximal the needle body has a diameter approximately equal to or slightly larger than a needle body diameter, and a second end of the tapered transition portion opposite the first end has a diameter equal to or slightly larger than a suture thread diameter, and wherein the tapered transition portion provides a smooth transition from the first end to the second end thereof. The tapered transition portion may be flexible. The tapered transition portion may be formed of a cyanoacrylate, or any other suitable biocompatible material.

According to certain embodiments, the needle body may be curved or straight. Moreover, the needle tip portion is formed to become gradually thicker from the needle tip to the proximal end thereof. The needle tip may be sharp or blunt.

According to certain embodiments, the needle tip portion and curved trunk portion are formed of a steel wire, a martensitic stainless steel, or an austenitic stainless steel, or any other suitable biocompatible material.

According to certain embodiments, the needle tip portion, the curved trunk portion, and/or the entire suture needle may be non-hollow. According to certain embodiments, the needle tip portion, the curved trunk portion, and/or the entire suture needle may be hollow.

According to certain embodiments, the needle body may have a substantially circular cross-section having a non-smooth profile. In one or more embodiment(s), the suturing needle comprises at least one of an oval, a triangular, a square, a rectangular, a pentagonal, a hexagonal, a heptagonal, an octagonal, a nonagonal, a decagonal, or a polygonal having more than ten sided, cross-section. In one or more embodiment(s), the suturing needle comprises multiple varied cross sections, including any combinations of the foregoing cross-sections. The non-smooth surface may comprise 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, about 150, about 200, about 250, about 300, about 350, about 400, about 450, or about 500 equally spaced circumferential indentations forming ridges. In one or more embodiment(s) the non-smooth surface may comprise 2, 3, 4, 5, 6, 7, 8, 9, 10, 11, 12, 13, 14, 15, 16, 17, 18, 19, 20, about 25, about 30, about 35, about 40, about 45, about 50, about 60, about 70, about 80, about 90, about 100, about 150, about 200, about 250, about 300, about 350, about 400, about 450, or about 500 non-equally spaced indentations forming ridges, or any number in between. In one or more embodiments, the non-smooth surface may comprise milli-indentations, micro-indentations, or nano-indentations. In one or more embodiments, the ridges have a convex shape. In one or more embodiments, the ridges have a flat peak, a pointed peak, a rounded peak, or a bumpy peak. In one or more embodiments, the peaks are smooth, rough, textured, ribbed, tacky, or polished.

According to certain embodiments, the circumferential indentations may be angled on the needle body to form a spiral thereon. The spiral may be right-handed or left-handed. Moreover, the needle body may comprise at least two regions of circumferential indentations having different angles on the curved needle body.

According to certain embodiments, the suture thread may be mono-filamentary or multi-filamentary. The suture thread may be non-bioabsorbable or bioabsorbable. The suture thread may comprise at least one region of tissue retaining structures, such as barbs. According to certain embodiments, the at least one region of tissue retaining structures may be distal from the tapered ramp.

The present disclosure further provides a method of suturing tissues using a rotational needle driver, the method comprising: (a) providing a suture needle comprising A suturing needle comprising: a needle tip portion having a needle tip at a distal end for piercing tissue; a needle body having a distal end connected to a proximal end of the needle tip portion; a suture thread attached to a proximal end of the needle body by a thread attachment component; and a tapered transition portion extending from the proximal end of the needle body and covering the thread attachment component and at least a portion of the suture thread; (b) grasping, between rollers of the roller needle driver, the suture thread or the tapered transition portion distal from the needle body; (c) loading the needle so that the rollers are positioned on the needle body without opening the rollers; (d) driving the needle through the tissues being subjected to suture; (e) moving the needle through the tissues without releasing the needle from the tissue; (f) grasping the suture needle at an end opposite from an end grasped in step (b); and repeating steps (d)-(f) until completion of the suturing process.

The system as described herein, both as to its configuration and its mode of operation will be best understood, and additional objects and advantages thereof will become apparent, by the following detailed description of a preferred embodiment taken in conjunction with the accompanying drawing.

When the word “invention” is used in this specification, the word “invention” includes “inventions”, that is, the plural of “invention”. By stating “invention”, the Applicant does not in any way admit that the present application does not include more the one patentable and non-obviously distinct invention and Applicant maintains that the present application may include more than one patentably and non-obviously distinct invention. The Applicant hereby asserts, that the disclosure of the present application may include more than one invention, and, in the event that there is more than one invention, that these inventions may be patentable and non-obvious one with respect to the other.

Further, the purpose of the accompanying abstract is to enable the U.S. Patent and Trademark Office and the public generally, and especially the scientists, engineers, and practitioners in the art who are not familiar with patent or legal terms or phraseology, to determine quickly from a cursory inspection the nature and essence of the technical disclosure of the application. The abstract is neither intended to define the full scope of the application, which is measured by the claims, nor is it intended to be limiting as to the scope of the disclosure in any way.

### BRIEF DESCRIPTION OF THE DRAWINGS

All of the figures depict preferred embodiments although other embodiments are contemplated, and the present disclosure is not limited to the embodiments shown.

FIG. 1 is an illustration of a suture needle having a tapered transition portion according to certain embodiments of the present disclosure.

FIGS. 2A-2C are illustrations of a suture needle having a tapered transition portion according to certain embodiments of the present disclosure, wherein FIG. 2A shows the rollers of a rotational driver engaging the tapered transition portion, FIG. 2B shows the rollers smoothly transitioning from the tapered transition portion over a thread attachment component, and FIG. 2C shown the rollers positioned on the needle body.

FIG. 3A is a curved needle having a tapered transition portion and a non-smooth profile according to certain embodiments of the present disclosure.

FIG. 3B is a cross-sectional view taken along line 1-1 of the curved suture needle shown in FIG. 3A according to certain embodiments of the present disclosure.

FIGS. 4A-4C are illustrations of alternative cross-sections of the suture needle having a non-smooth profile according to certain embodiments of the present disclosure.

FIG. 5 illustrates an alternative arrangement of the non-smooth surface according to certain embodiments of the present disclosure.

FIGS. 6A and 6B are illustrations of a curved suture needle, shown in partial cross-section, according to certain embodiments of the present disclosure held within a rotational needle driver, wherein FIG. 6A shows the needle in the usual perpendicular orientation passing behind the rollers of the driver, and FIG. 6B shows the needle in the usual perpendicular orientation passing in front of the rollers of the driver.

FIGS. 7A and 76B are illustrations of a curved suture needle according to certain embodiments of the present disclosure held within a rotational needle driver, wherein FIG. 7A shows the needle in a parallel orientation passing in front of the rollers of the driver, and FIG. 7B shows the needle in the an angled orientation relative to the rollers and passing in front of the rollers of the driver.

FIG. 8 illustrates an alternative arrangement of the non-smooth surface according to certain embodiments of the present disclosure.

### DETAILED DESCRIPTION

To provide an overall understanding of the disclosure, certain illustrative embodiments and examples will now be described. However, it will be understood by one of ordinary skill in the art that the same or equivalent functions and sequences may be accomplished by different embodiments that are also intended to be encompassed within the spirit and scope of the disclosure. The compositions, apparatuses, systems and/or methods described herein may be adapted and modified as is appropriate for the application being addressed and that those described herein may be employed in other suitable applications, and that such other additions and modifications will not depart from the scope hereof.

As used in the specification and claims, the singular forms “a”, “an” and “the” include plural references unless the context clearly dictates otherwise. For example, the term “material” may include a plurality of materials unless the context clearly dictates otherwise. As used in the specification and claims, singular names or types referenced include variations within the family of said name unless the context clearly dictates otherwise.

Certain terminology is used in the following description for convenience only and is not limiting. The words “lower,” “upper,” “bottom,” “top,” “front,” “back,” “left,” “right” and “sides” designate directions in the drawings to which reference is made, but are not limiting with respect to the orientation in which the various parts of the needle or any assembly of them may be used.

Swaged suturing needles typically comprise attachment of a suture thread to an end of a needle body. These needles have been designed for use with standard needle drivers, which clamp the needle between two flat tips and hold the needle in a static position.

A new generation of devices that move the needle through the tissues by the coordinated rotation of two rollers provide a dynamic relationship between the needle and the needle driver, even when the needle is clamped between the rollers. One of the great advantages of this dynamic relationship is that the needle can be driven in multiple planes of rotation, such as perpendicular to the long axis of the driver with the tip pointing up or down (standard), parallel with the long axis of the driver with the tip pointing straight back towards the back of the device or straight forward away from the device (non-standard), and many angles in between. This dynamic relationship is desirable for the surgeon as it enables placement of the needle at non-traditional angles, permitting suturing of difficult anatomical regions or situations, such as restricted, deep and less accessible locations.

According to certain embodiments, a needle comprising a needle tip portion having a needle tip at a distal end for piercing tissue, a straight or curved trunk portion having a distal end connected to a proximal end of the needle tip portion; and a thread attachment portion connected to a proximal end of the trunk portion for connecting a suturing thread, wherein the needle tip portion is formed to become gradually thicker from the needle tip to the proximal end thereof, and wherein the trunk portion is formed to have a substantially circular cross-section having a non-smooth profile. In certain embodiments, the needle may have a smooth profile. In one or more embodiment(s), the needle comprises at least one of a circular, an oval, an ellipsoidal, an oblong, a semi-circular, a triangular, a square, a rectangular, a pentagonal, a hexagonal, a heptagonal, an octagonal, a nonagonal, a decagonal, or a polygonal having more than ten sided, cross-section. In one or more embodiment(s), the suturing needle comprises multiple varied cross sections, including any combinations of the foregoing cross-sections.

Some of these suture needles can be used for medical purposes (e.g., suturing). For example, some of these suture needles can be used in transcatheter suturing, transcatheter intracardiac (or another body organ) suturing, and other flexible platform applications (e.g., endoscopic suturing, colonoscopic suturing). For example, rigid shaft embodiments, as commonly used for laparoscopic, video-assisted thoracoscopic surgery (VATS) and robotic surgery can be used to fundamentally transform suture-based tissue approximation/anchoring from a laborious task to a swift semi-automated or fully-automated endeavor. For example, some of these suture needles can be used for an open surgery, a minimally invasive surgery, a laparoscopic surgery, or an end effector robotic surgery. As such, the some of these suture needles can be used for manual surgery or automated surgery. Some examples of surgeries where some of these suture needles can be employed include laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, hysterectomy surgery, dental surgery, urological surgery, brain surgery, or bariatric surgery, or among many others in human (e.g., between newborn until 120 years old, male, female) or animal (e.g., mammal, birds, fish, land animals) applications.

Note that some of these suture needles can be employed in medical or non-medical settings, whether on an object is animate or inanimate. For example, the object, when animate, can include a tissue, an organ, a body part, whether of human or animal, or others. For example, the tissue can be a muscle tissue, a bone tissue, a nerve tissue, an organ tissue, or others. For example, the object, when inanimate, can include a medical device, a prosthesis, an implantable, a machine, a surgical instrument, or others. For example, some of the non-medical setting can include garment making, fabric stitching, knot applications, sowing, shoe making, or others.

Current swaged suture needles generally include a lip or step-up in the transition from the suture thread to the thread attachment connector, and another lip or step-up in the transition from the thread attachment component to the needle body. Alone or in combination, these transitions pose a problem for the rollers of a rotational needle driver, as the suture thread may be damaged by the rollers, or conversely, the transitions may damage the rollers of the needle driver. For example, the rollers may roll past the thread attachment component of the needle and may clamp down on that region and damage/cut the suture thread.

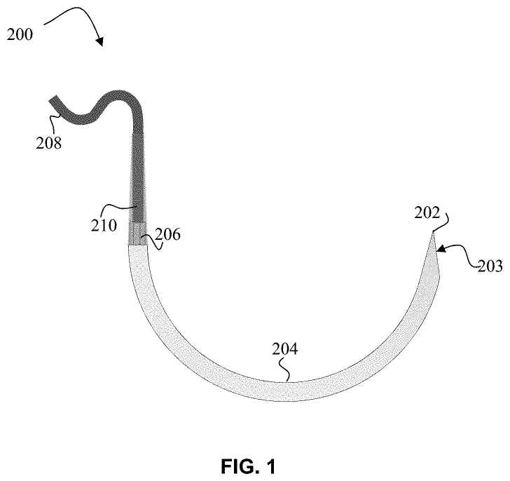

The present disclosure overcomes these deficiencies by incorporating a novel tapered transition portion that provides a smooth transition from the suture thread to the needle body. With reference to FIG. 1, a suture needle 200 of the present disclosure may comprise a tapered transition portion 210. This protective leading segment immediately follows the distal end of the needle body 204, such as covering the thread attachment component 206 of the needle and extends over a proximal portion of the suture thread 208. In certain embodiments, there is no or minimal transition between the trunk portion 204 of the needle and the thread attachment portion 206. In such embodiments, the transition portion 210 provided a taper from the trunk portion 204 and past the thread attachment portion 206. In certain embodiments, the tapered transition portion 210 includes a proximal side that overlaps the trunk portion 204 in a direction of the needle tip and includes a distal side that extends to a point overlapping the suture to form a taper. In certain embodiments, the taper is gradual. In certain embodiments the taper is steep. In certain embodiments, the swage is tapered. In certain embodiments, the swage is non-tapered.

The tapered transition portion 210 may extend over the suture material a distance from the end of the needle body 204 of the needle, such as from about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 2 cm, about 3 cm, about 4 cm, about 5 cm, about 6 cm, about 7 cm, about 8 cm, about 9 cm, about 10 cm, about 15 cm, about 20 cm, or any distance in between. Moreover, the tapered transition portion 210 may be formed of a flexible material that may assist in loading the needle onto the needle driver, but may have more rigidity than the suture material, such as to aid in suture management during knot tying. The length of the tapered transition portion 210 may be determined by the optimum balance between the minimal length needed by the surgeon to grab the needle by the “ramp” when inserting it or withdrawing it to a surgical site, such as through a laparoscopic port, and the maximal length that can feasibly allow tapering and enough flexibility to go through tissue following the needle path without causing tissue trauma.

The tapered transition portion 210 may be a cyanoacrylate or another suitable coating (or filament) material, such as, for example, another biocompatible material, that may be built up from a diameter of the suture material 208 to a diameter of the needle body 204 of the needle 200. The tapered transition portion 210 may be formed by a shrink-wrapped procedure wherein a preform of the tapered region is positioned over the suture thread and swage to abut an end of the trunk portion of the needle, and heat may be applied to form the preform on the needle, thus forming the tapered transition portion 210. As such, this protective segment or tapered transition portion 210 may be shaped as a “conical ramp” that allows the rollers to be rolled from the tapered transition portion 210 “up the ramp” in a continuous rolling motion until reaching the needle body 204 of the needle 200 without hitting the blunt swage end of the needle, and thus avoiding potential damage to the needle, rollers, and/or suture material. In certain embodiments, the tapered transition portion may comprise a magnetic material or a magentizable material. In certain embodiments, the suture material may comprise a magnetic material or a magnetizable material.

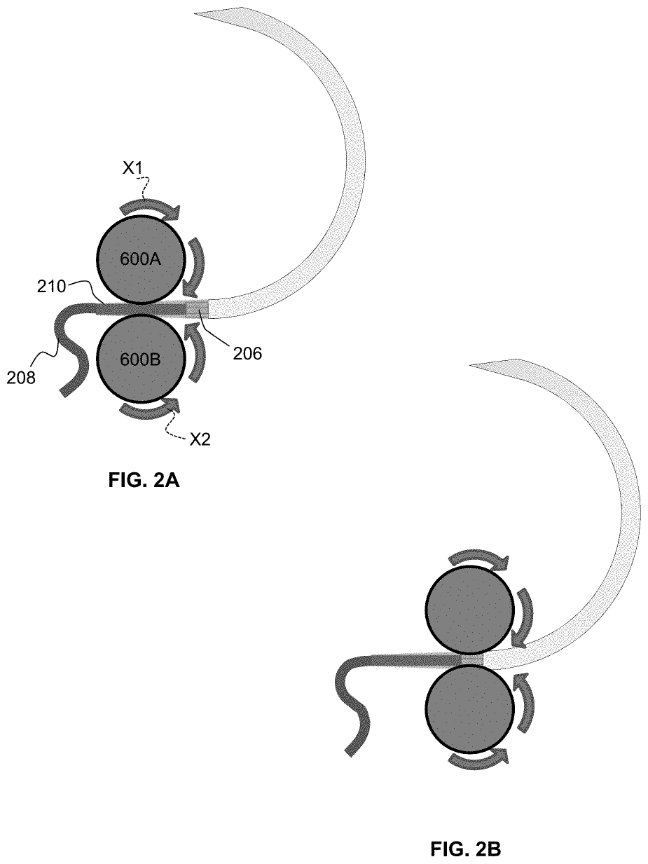

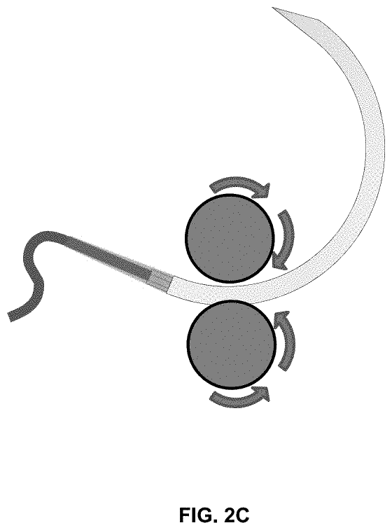

This creates a procedural benefit and efficiency by loading the needle onto the needle driver to a position ready for suturing all in a single step, as shown in FIGS. 2A-2C. The rollers 600A and 600B may be closed over the suture material 208 and may rotate in opposite directions (x1 and x2, respectively) to advance the needle forward, shown as the transition from the needle position in FIG. 2A to FIG. 2C. As the needle advances between the rollers of the needle driver, the rollers smoothly advance the needle from the suture material to the tapered transition portion 210 (FIG. 2A). Without opening the rollers, the rollers may continue to rotate to advance the needle along the tapered transition portion 210 over the thread attachment component 206 (FIG. 2B).

In the absence of the tapered transition portion 210, this would represent a step or lip on the needle that may be damaged by the rollers, or conversely, that may damage the rollers. Accordingly, in the absence of the tapered transition portion 210, the clinician would have to use a grasper to hold/stabilize the needle, then open the needle driver to release the suture it was grasping during needle insertion and then grasp the needle with the needle driver once positioned over the needle body in preparation for suturing. In a laparoscopic setting each of these steps may require time, effort, coordination and maybe even repetition due to the lack of depth perception (2D visualization systems used mostly) and decreased dexterity, fulcrum effect of port, etc. Finally, as shown in FIG. 2C, the rotation of the rollers advances the needle so that it is positioned between the rollers and properly loaded for suturing without ever opening the rollers. These motions may be reversed to unload the needle, such as to remove the needle from a suture site (e.g., from the abdomen or from a laparoscopic surgical site).

With reference to FIG. 1A, when used with a rotational needle driver, such as discussed above, the rollers may roll past the thread attachment portion 206 of the needle 200 and may clamp down on that region and damage/cut the suture thread. Thus, according to certain embodiments, the suture needle of the present disclosure may comprise a tapered transition portion 210, such as shown in FIG. 2A. This protective leading segment immediately follows the distal end of the trunk portion 204 of the needle, such as covering the thread attachment portion 206 of the needle, and extends over a proximal portion of the suture material.

With reference to FIG. 1, a suture needle 200 of the present disclosure generally comprises a needle tip 202 for piecing a tissue. The needle tip 202 is positioned at a distal end of a needle tip portion 203, wherein the needle tip portion becomes gradually thicker from the needle tip to a proximal end thereof. Thus, the needle tip portion may be a tapered tip wherein the needle tip portion is round and tapers smoothly to a point. Alternatively, the needle tip portion may be triangular as shown in FIG. 1, and may have a sharpened cutting edge on the inside or on the outside, or may have a “trocar point” or “tapercut” whereby the needle body is round and tapered, but ends in a small triangular cutting point. Alternatively, the needle tip may be a blunt point, such as for suturing friable tissues, or may have a needle tip portion that includes “side cutting” or “spatula points” whereby the needle is flat on top and bottom with a cutting edge along the front to one side (these are typically used for eye surgery).

Continuous with the proximal end of the needle tip portion 203 is the needle body 204 having the thread attachment component 206 positioned at a distal end thereof. The needle body 204 is shown to have a curve in the drawings. Exemplary curves include at least half curved or ski, ¼ circle, ⅜ circle, ½ circle, such as shown in the figures, ⅝ circle, and compound curve. Alternatively, the needle body may be straight, such that the present disclosure may comprise a straight needle.

The material configuring the suture needle is not limited, and may comprise a metal or a metal alloy, such as, for example, a biocompatible metal or metal alloy. In certain embodiments, the suture needle comprises any of steel wire, a martensitic stainless steel, or an austenitic stainless steel. In certain embodiments, the suture needle is comprised of a magnetic or magnetizable material. When formed of steel wire or martensitic stainless steel, the needle may be hardened by thermal treatment. Other processes when forming or finishing the needle may be siliconization, coating with any number or combination of biocompatible coating materials or lubrication with any number or combination of biocompatible lubricating agents, among other processes.

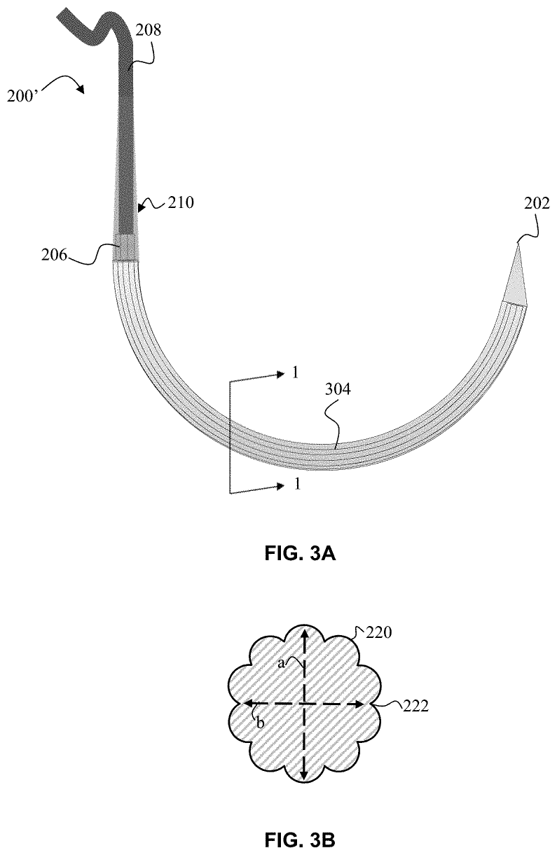

As shown in FIGS. 1 and 3A, for example, the suture thread 208 may be swaged to the needle 200 by the thread attachment component 206. Accordingly, the needle is generally an atraumatic needle, i.e., eyeless needle, having a suture material or thread attached at an end by swaging whereby the suture material is inserted into a channel at the blunt end of the needle, such as into the thread attachment component 206, which is then deformed to a final shape to hold the suture and needle together. The needle may be permanently swaged to the suture material or may be designed to come off the suture material with a sharp straight tug. These “pop-offs” are commonly used for interrupted sutures, where each suture is only passed once and then tied.

The suture material or thread may be mono-filamentary, i.e., formed of a single filament, or multi-filamentary, i.e., formed from a combination of two or more filaments, e.g., three filaments arranged in a braided fashion. The suture thread has a length, where that length is typically at least 5 inches, or at least 10 inches, or at least 15 inchers, or at least 20 inches. The suture thread will typically have two ends, which may be described as a deployment end and/or a trailing end. In such a case, the deployment end of the suture thread is that end that first enters tissue, adjacent to the needle, such as connected via the thread attachment component to the distal end of the needle body of the needle. Alternatively, the suture material may be looped, such that each of the two free ends are connected to the needle by the thread attachment component.

The thread 112 can be a suture, which can be non-absorbable or absorbable of various gauges. The thread 112 can include silk, cotton, fabric, nylon, polyester, silver, copper, Dacron, rubber, silicon, plain or chromic catgut, polyglycolide, polydioxanone, monocryl, polypropylene, triclosan, caprolactone, polymer, glycolide, l-lactide, p-dioxanone, trimethylene carbonate, ε-caprolactone, stainless steel, ceramic, glass, leather, or other natural or artificial materials. The thread 112 is solid, but can be perforated. The thread 112 is internally dense, but can be hollow. The thread 112 can be rigid, semi-rigid, elastic, resilient, or flexible. For example, the thread 112 can bend about 90 degrees or less (e.g., inclusively between or about 90, 80, 70, 60, 50, 40, 30, 20, or 10 degrees) or more (e.g., inclusively between or about 90, 100, 110, 120, 130, 140, 150, 160, 170, or 180 degrees). The thread 112 can have a cross-section that is closed-shaped (e.g., O-shape, D-shape, O-shape, square, rectangle, triangle, polygon) or open-shaped (e.g., U-shape, C-shape, V-shape), whether symmetrical or asymmetrical.

The suture material or thread may be bioabsorbable, such that after introduction into a tissue it is broken down and absorbed by the body. Typically, the degradation process is at least partially mediated by, or performed in, a biological system. Accordingly, bioabsorbable refers to a chain scission process by which a polymer chain is cleaved through various mechanisms, including, for example, by chemical reaction (e.g., hydrolysis, oxidation/reduction, enzymatic mechanisms or a combination of these) or by a thermal or photolytic process. Bioabsorbable suture material may include polymers such as polyglycolic acid, copolymers of glycolide and lactide, copolymers of trimethylene carbonate and glycolide with diethylene glycol (e.g., MAXON™, Tyco Healthcare Group), terpolymer composed of glycolide, trimethylene carbonate, and dioxanone (e.g., BIOSYN™[glycolide (60%), trimethylene carbonate (26%), and dioxanone (14%)], Tyco Healthcare Group), copolymers of glycolide, caprolactone, trimethylene carbonate, and lactide (e.g., CAPROSYN™, Tyco Healthcare Group). In certain embodiments, the bioabsorbable suture material may comprise or include any other polymer useful for suturing applications that currently exists or that may be developed in the future.

Alternatively, the suture material or thread may be non-degradable, such that it is not degraded by chemical, thermal, or photolytic process. Non-degradable suture material includes polyamide (also known as nylon, such as nylon 6 and nylon 6.6), polyester (e.g., polyethylene terephthlate), polytetrafluoroethylene (e.g., expanded polytetrafluoroethylene), polyether-ester such as polybutester (block copolymer of butylene terephthalate and polytetra methylene ether glycol), polyurethane, metal alloys, metal (e.g., stainless steel wire), polypropylene, polyethelene, silk, and cotton. Sutures made of non-degradable suture material are particularly suitable for applications in which the suture is meant to remain permanently or is meant to be physically removed from the body. In certain embodiments, the non-degradable suture material may comprise or include any other polymer useful for suturing applications that currently exists or that may be developed in the future.

The suture material or thread may comprise a coating or agent applied to a surface thereof that may affect would healing, such as a coating material, wound healing agent, antimicrobial agent, antibacterial agent, growth factor, adhesive, sealant, blood product, blood component, preservative, anti-adhesive, protein, polysaccharide, peptide, genetic material, viral vector, nucleic acid, nucleotide, plasmid, lymphokine, radioactive agent, metal, alloy, salt, growth factor, growth factor antagonist, cell, hydrophobic agent, hydrophilic agent, immunological agent, anti-colonization agent, and combinations thereof. The suture material or thread may comprise a coating or agent applied to a surface thereof that may enhance the surgeon's ability to accurately suture, such as colorants, dyes, ultraviolet absorbers, ultraviolet stabilizers, photochromic agents, diagnostic agent, imaging agent, radiopaque agent, or combinations thereof.

The suture material or thread may have a diameter indicated by a cross-sectional dimension. This diameter may be determined at a location along the suture where there are either no barbs, or the barbs that are present are pushed against the suture body so that they are flush with the surface of the suture body. The suture may have no barbs or may have barbs along all or only a portion of the suture length. According to certain embodiments, the suture may have at least one barbed region positioned distal from the thread attachment component. Such positioning may allow the presently disclosed suture needle to be backed out of a suture site, wherein the suture material adjacent the needle would not have barbs and would easily slide back through the tissue. In some embodiments the tapered transition portion covers the barbs on the suture, thereby allowing the suture needle to be backed out of the suture sight. More distally located barbed suture may remain securely in place once positioned in the tissue.

The suture may have a generally circular cross-sectional shape, or may have a non-circular shape, e.g., polygonal such as 3-sided (triangular), or 4-, 5- or 6-sided (hexagonal) sided. The cross section of the suture body may have an oval, an ellipsoid, an oblong, or a semi-circular appearance.

Suture sizing is based upon diameter. The United States Pharmacopeia (“USP”) designation of suture size runs from 0 to 7 in the larger range and 1-0 to 11-0 in the smaller range; in the smaller range, the higher the value preceding the hyphenated zero, the smaller the suture diameter. Under the USP nomenclature system, the actual diameter of a suture will depend on the suture material, so that, by way of example, a suture of size 5-0 and made of collagen will have a diameter of 0.15 mm, while sutures having the same USP size designation but made of a synthetic absorbable material or a non-absorbable material will each have a diameter of 0.1 mm. The selection of suture size for a particular purpose depends upon factors such as the nature of the tissue to be sutured and the importance of cosmetic concerns; while smaller sutures may be more easily manipulated through tight surgical sites and are associated with less scarring, the tensile strength of a suture manufactured from a given material tends to decrease with decreasing size. It is to be understood that the suture materials for use with the suture needles disclosed herein include without limitation 7, 6, 5, 4, 3, 2, 1, 0, 1-0, 2-0, 3-0, 4-0, 5-0, 6-0, 7-0, 8-0, 9-0, 10-0 and 11-0. It is to be understood that a variety of suture lengths may be used with the suture needles described herein.

The needle body may have any profile known in the art, such as circular, oval, triangular, and the like. The needle body have a smooth surface. According to certain embodiments, the needle body may be a non-smooth profile. For example, when trying to drive needle through a tissue using a rotational needle drive, a smooth surface of the needle body may allow the needle to deviate out of an initial chosen plane of rotation, especially for needle bodies having an oval shape. Moreover, it is not possible to securely position these needles in a non-standard angle. Accordingly, an object of the present disclosure is to provide suture needles that may perform optimally in roller needle drivers and may incorporate features that enable multi-planar needle driving.

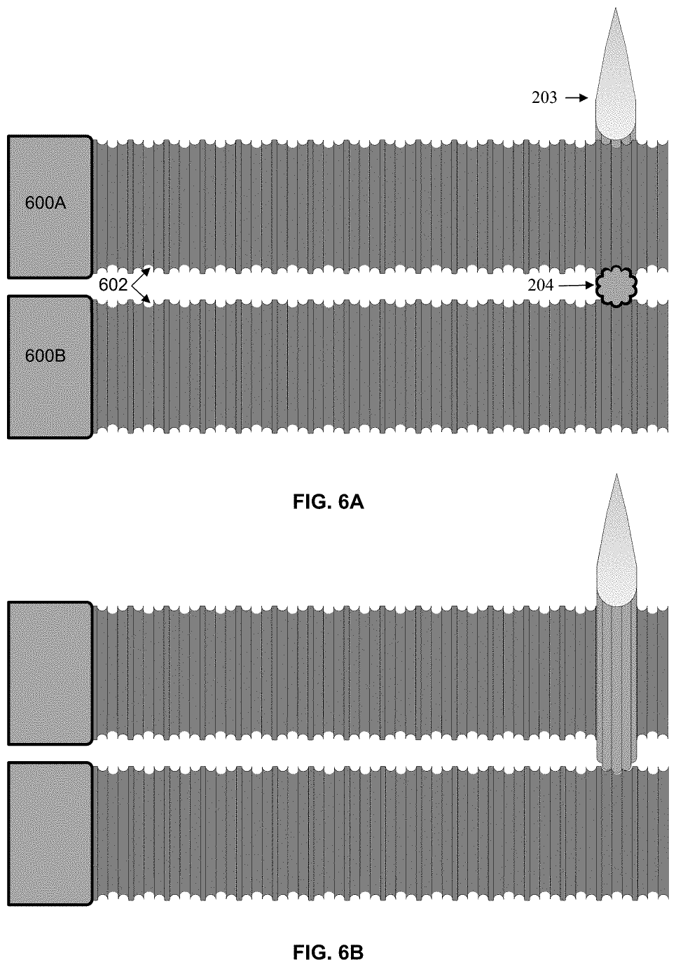

Rotational needle drivers generally comprise an actuator portion having linear and rotational motion systems and an interactive portion configured to interact with the suture needle. Each interactive portion comprises a first extended member and a second extended member which approximate a pair of chopsticks that may be opened and closed. Each extended member comprises a main needle-grasping portion having grooves, such as grooves 602 on each of a first 600A and second 600B roller as shown in FIG. 6A. The suture needle of the present disclosure are designed to include ridges that mate with these grooves 602 of the rollers so that a secure connection between the needle and the rollers may be achieved.

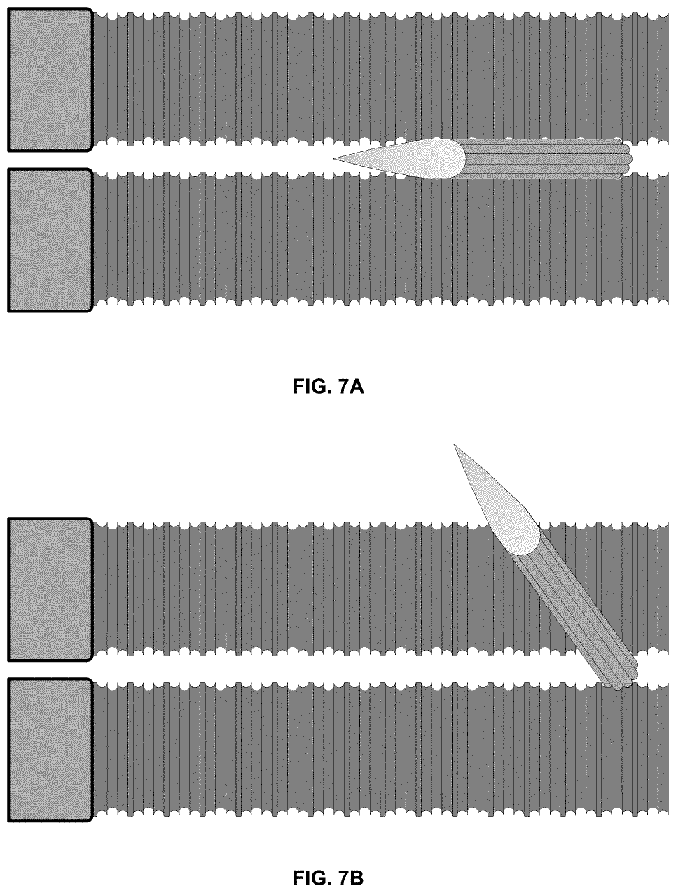

As shown in FIGS. 6A and 6B, the needle may be grasped between the rollers to position the needle perpendicular to the rollers, passing behind the rollers of the driver as in FIG. 6A or in front of the rollers as in FIG. 6B. The unique arrangement of ridges on the suture needle further provide for grasping between the two rollers at non-standard angles, such as parallel with the rollers as shown in FIG. 7A or even oblique to the rollers as shown in FIG. 7B, or any of a number of angles therebetween.

When the suturing procedure begins, the suture needle having a non-smooth surface configured to interact with the grooves of the two rollers is grasped at a desired angle. The surgeon may press an actuator on the needle driver that brings the rollers closer so that they may grasp the needle and may lock the rollers in position exerting the necessary force to maintain the needle still. Once the needle is tightened, the actuator may create a rotational movement of the rollers, such as shown in FIG. 2A, wherein the rollers rotate in directions opposite from each other to affect movement of the needle in either of two directions. Thus, the surgeon may drive the needle through the tissue as the rollers rotate. When the desired rotation is completed, the surgeon releases the locking mechanism. This method is repeated on the other tissue that is to be joined to the tissue already perforated by the needle, and each step above is repeated as many times as stitches have to be performed.

The design of the roller needle driver permits rotation of a curved suture needle without requiring a rotational motion of the wrist. Therefore, the instrument can be held in a position that may not allow wrist rotation and still carry out its function. In some situations, the structures to be sutured lie very deep. Doing surgery on the vertebral column of very obese patients is one such situation. For example, if the dura mater (a membrane that protects and envelops the spinal cord) is cut it needs to be sutured. The dura mater lies within a deep constricted space. In a very obese patient, the additional thickness of the adipose (fatty) tissue makes the dura lie even deeper from the surface. The surgeon may have to lean towards the patient and rotate the arm bearing the needle driver. This is necessary to get his forearm in a vertical position so he can maneuver the instrument properly. The needle driver used with a suture needle as disclosed herein may be operated in such a position.

According to certain embodiments, the needle 200′ of the present disclosure may comprise a non-smooth surface. With reference to FIG. 3B, a cross-sectional view of the curved needle body taken along line 1-1 of FIG. 3A shows this region to have a circular shape with a non-smooth profile. This non-smooth profile can be achieved by forming indentations 222 in the outer circumferential periphery of the needle body. As shown, these indentations may be evenly spaced, and may form protrusions 220, such as the smooth convex protrusions shown in FIG. 3B.

These indentations 222 and/or protrusions 220 create longitudinal ridges along the length of the needle body 304 and may be uniquely designed and configured to fit within the grooves of the rollers of a rotational needle driver. That is, these ridges may fit within the grooves of the rollers of the needle driver and act like rails to secure the orientation of the needle in the groove while the needle is being driven by the rotation of the rollers. Because the needle 200′ has ridges evenly spaced about its round cross-section, the needle can be placed in any of a number of angles on the rollers. See for example the rollers (600A, 600B) shown in FIGS. 6A-7B that are grasping a needle along the needle body 304, wherein the ridges (indentations 222 and protrusion 220) match the grooves 602 of the plurality of rollers independent of the orientation of the needle. In certain embodiments, the ridges are unevenly spaced. In certain embodiments, the ridges have different heights. In certain embodiments, the ridges have different orientations.

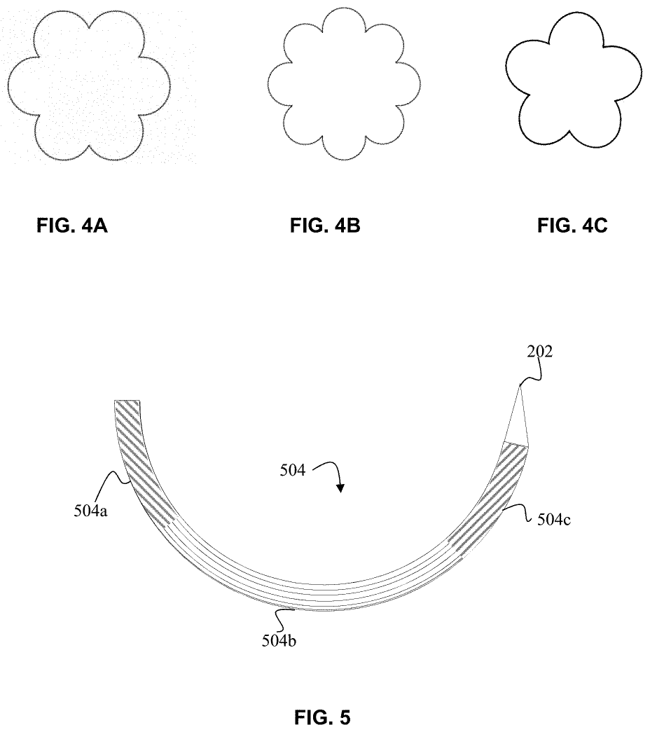

With reference to FIG. 3B, the depth of the indentations 222 (difference in diameter a and b of the needle body 304), and their spacing about the circumference of the needle body defines the ridges. These dimensions may be selected so that the ridges match the depth and spacing of grooves on the rollers of a rotational needle driver (such as shown in FIGS. 6A-7B). The specific design shown in FIG. 3B is exemplary only, as many other designs and configurations are possible, such as shown in FIGS. 4A-4C which include different numbers of indentations and thus different numbers of convex protrusions (ridges). Moreover, while the protrusions or ridges are shown to have a generally convex shape without any sharp angles (i.e., no pointed edges), other configurations are possible and within the scope of the present disclosure.

The ridges formed on the suture needle generally extend along a full longitudinal length of the needle body 304, as shown in FIG. 3A. According to certain embodiments, the indentations 222 may be formed about a circumference of the needle body at an angle, thus forming ridges (i.e., protrusions 220) that may spiral about the outer circumference of the needle body 304 of the needle 200′. The angle may be selected so that the ridges have a right-handed or left-handed spiral. Moreover, as shown in FIG. 5, the angle of the ridges may be changed one or more times along a length of the needle body 504. For example, the ridges may have a right-handed angle (504a), no angle (504b), or a left-handed angle (504c). The specific angle, size of the angled region, and placement thereof may be selected based on suture procedure requirements.

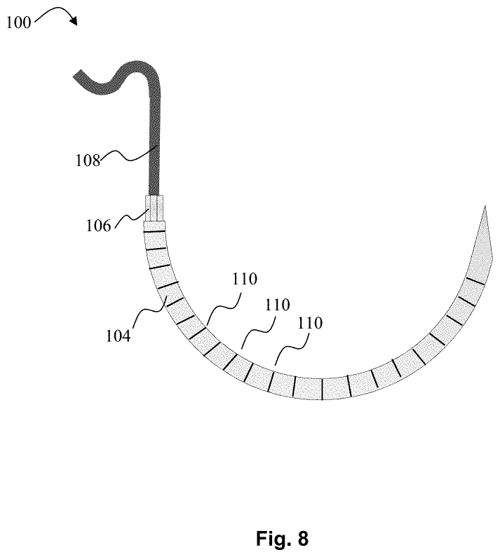

In certain embodiments, for example, as shown in FIG. 8, the suture needle 100 includes indentations and/or ridges 110 that extend circumferentially around the suture needle body, such that they provide improved traction for the rollers of the needle driver to move the suture needle through dense, compact, or hard material. In certain embodiments, the circumferential ridges 110 may be closely spaced one from the other. In certain embodiments, the circumferential ridges 110 may be spaced apart one from the other. In certain embodiments, the distance between the circumferential ridges 110 is fixed. In certain embodiments, the distances between circumferential ridges 110 varies from ridge to ridge. In certain embodiments, the distances between circumferential ridges 110 varies according to a predetermined pattern, formula, or algorithm. In certain embodiments, one or more of the rollers of the needle driver include indentations or ridges that run from a proximal end to a distal end of one or more of the grooves of the rollers. In certain of the embodiments, the indentations or ridges in the grooves of the rollers mate or interleave with the circumferential ridges 110 or indentations on the suture needle. This provides the rollers with improved traction for the rollers of the needle driver to move the suture needle through dense, compact, or hard material.

In certain embodiments, the suture needle includes ridges that extend along only a portion of the needle trunk. In certain embodiments, the suture needle includes ridges that begin at a first portion and end at a second portion of the needle trunk. In certain embodiments, the suture needle includes multiple discontinuous areas having ridges with intervening non-ridged areas. In certain embodiments, the suture needle includes certain areas with at least one of ridges, a groove surface, a roughened surface, a smooth surface, a bumpy surface, a ribbed surface, a tacky surface, or a polished surface. In certain embodiments, the suture needle includes certain areas with at least one of a ridged surface, a grooved surface, a roughened surface, a smooth surface, a bumpy surface, a ribbed surface, a tacky surface, or a polished surface, and other areas with at least one different surface. In certain embodiments, the ridged and non-ridged sections, or any combinations thereof may each be of any particular length, width, order, or orientation. In certain embodiments, the ridged surface, the grooved surface, the roughened surface, the smooth surface, the bumpy surface, the ribbed surface, the tacky surface, or the polished surface sections, or any combinations thereof may each be of any particular length, width, order, or orientation. In certain embodiments, there may be more than one type of non-ridged surface, such as, for example, a ridged surface, a grooved surface, a roughened surface, a smooth surface, a bumpy surface, a ribbed surface, a tacky surface, or a polished surface. For example, you can have ridged sections, smooth sections and otherwise textured non-ridged sections combined in the same needle trunk for any type of application.

In certain embodiments, a suture needle may comprise a smooth tip section, ridged body and textured-non-ridged portion near the swage end that could induce some slight vibration captured by a robotic needle driver to improve haptic feedback to the surgeon on when to pull the needle out of the tissue to minimize tissue trauma, particularly in regions where tissue visibility or maneuverability is quite limited or tissues are very prone to tearing if the needle is pulled from the tissues too soon.

Features or functionality described with respect to certain example embodiments may be combined and sub-combined in and/or with various other example embodiments. Also, different features and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

As used herein, a term “about” or “substantially” refers to a +/−10% variation from a nominal value/term. Such variation is always included in any given value/term provided herein, whether or not such variation is specifically referred thereto.

In particular, a suture needle 200 can be used for an open surgery, a minimally invasive surgery, a laparoscopic surgery, or an end effector robotic surgery. As such, the suture needle 200 can be used for manual surgery or automated surgery. Some examples of surgeries where the suture needle 200 can be employed include laparoscopic surgery, robotic surgery, video-assisted or unassisted thoracoscopic surgery, arthroscopic surgery, natural orifice surgery, endoscopic surgery, gynecologic surgery, cardiac surgery, colorectal surgery, pulmonary surgery, gastric bypass surgery, hysterectomy surgery, dental surgery, urological surgery, brain surgery, or bariatric surgery, or among many others in human (e.g., between newborn until 120 years old, male, female) or animal (e.g., mammal, birds, fish, land animals) applications. However, note that the suture needle 200 can also be applied to non-medical applications, such as garment making, fabric stitching, knot applications, sowing, shoe making, or others.

Any component described herein can include a material suitable for a medical use. The material can be, flexible, elastic, or resilient. The material can be suitable to be disinfected, sterilized, or sanitized, which can be with a hot steam, an autoclave, or others. For example, the material can include plastic, metal, rubber, shape memory, fabric, foam, or others.

The device and system of the present disclosure has been described with specific reference to certain drawings and various embodiments, but may, however, be embodied in many different forms and should not be construed as necessarily being limited to only embodiments disclosed herein. Rather, these embodiments are provided so that this disclosure is thorough and complete, and fully conveys various concepts of this disclosure to skilled artisans.

Note that various terminology used herein can imply direct or indirect, full or partial, temporary or permanent, action or inaction. For example, when an element is referred to as being “on,” “connected” or “coupled” to another element, then the element can be directly on, connected or coupled to the other element or intervening elements can be present, including indirect or direct variants. In contrast, when an element is referred to as being “directly connected” or “directly coupled” to another element, there are no intervening elements present.

Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Similarly, as used herein, various singular forms “a,” “an” and “the” are intended to include various plural forms as well, unless context clearly indicates otherwise. For example, a term “a” or “an” shall mean “one or more,” even though a phrase “one or more” is also used herein.

Moreover, terms “comprises,” “includes” or “comprising,” “including” when used in this specification, specify a presence of stated features, integers, steps, operations, elements, or components, but do not preclude a presence and/or addition of one or more other features, integers, steps, operations, elements, components, or groups thereof. Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

Additionally, although terms first, second, and others can be used herein to describe various elements, components, regions, layers, or sections, these elements, components, regions, layers, or sections should not necessarily be limited by such terms. Rather, these terms are used to distinguish one element, component, region, layer, or section from another element, component, region, layer, or section. As such, a first element, component, region, layer, or section discussed below could be termed a second element, component, region, layer, or section without departing from this disclosure.

Also, unless otherwise defined, all terms (including technical and scientific terms) used herein have the same meaning as commonly understood by one of ordinary skill in an art to which this disclosure belongs. As such, terms, such as those defined in commonly used dictionaries, should be interpreted as having a meaning that is consistent with their meaning in a context of a relevant art and should not be interpreted in an idealized or overly formal sense unless expressly so defined herein.

In addition, features described with respect to certain example embodiments may be combined in or with various other example embodiments in any permutational or combinatory manner. Different features or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

Although preferred embodiments have been depicted and described in detail herein, skilled artisans know that various modifications, additions, substitutions and the like can be made without departing from spirit of this disclosure. As such, these are considered to be within the scope of the disclosure, as defined in the following claims.

1. A suturing needle comprising:
a needle tip portion having a needle tip at a distal end for piercing tissue;
a needle body having a distal end connected to a proximal end of the needle tip portion;
a suture thread attached to a proximal end of the needle body by a thread attachment component; and
a tapered ramp extending from the proximal end of the needle body and covering the thread attachment component and at least a portion of the suture thread.

2. The needle of claim 1, wherein a first end of the tapered ramp proximal the needle body has a diameter equal to a needle body diameter, and a second end of the tapered ramp opposite the first end has a diameter equal to a suture thread diameter, and wherein the tapered ramp provides a smooth transition from the first end to the second end thereof.

3. The needle of claim 1, wherein the tapered ramp is flexible.

4. The needle of claim 1, wherein the tapered ramp is formed of a cyanoacrylate

5. The needle of claim 1, wherein the needle tip portion is formed to become gradually thicker from the needle tip to the proximal end thereof.

6. The needle of claim 1, wherein the suture thread is mono-filamentary or multi-filamentary.

7. The needle of claim 1, wherein the suture thread is bioabsorbable.

8. The needle of claim 1, wherein the suture thread comprises at least one region of tissue retaining structures.

9. The needle of claim 8, wherein the tissue retaining structures are barbs.

10. The needle of claim 8, wherein the at least one region of tissue retaining structures are distal from the tapered ramp.

11. The needle of claim 1, wherein the needle body is straight.

12. The needle of claim 1, wherein the needle body curved.

13. The needle of claim 1, wherein the needle body is formed to have a substantially circular cross-section having a non-smooth profile.

14. The needle of claim 11, wherein the non-smooth profile comprises a plurality of equally spaced circumferential indentations extending longitudinally on the curved needle body.

15. The needle of claim 14, wherein the non-smooth profile comprises from 4 to 16 equally spaced circumferential indentations forming ridges having a convex shape.

16. The needle of claim 14, wherein the circumferential indentations extend along a full length of the curved needle body.

17. The needle of claim 16, wherein the circumferential indentations are angled on the curved needle body to form a spiral thereon.

18. The needle of claim 17, wherein the spiral is right-handed or left-handed.

19. The needle of claim 17, comprising at least two regions of circumferential indentations having different angles on the curved needle body.

20. A method of suturing tissues using a roller needle driver, the method comprising:
(a) providing a suture needle comprising: a needle tip portion having a needle tip at a distal end for piercing tissue; a needle body having a distal end connected to a proximal end of the needle tip portion; a suture thread attached to a proximal end of the needle body by a thread attachment component; and a tapered ramp extending from the proximal end of the needle body and covering the thread attachment component and at least a portion of the suture thread;
(b) grasping, between rollers of the roller needle driver, the suture thread or the tapered ramp distal from the needle body;
(c) loading the needle so that the rollers are positioned on the needle body without opening the rollers;
(d) driving the needle through a tissue being subjected to suture;
(e) moving the needle through the tissue without releasing the needle from the tissue;
(f) grasping the suture needle at an end exiting from the tissue through which the needle was moved; and
repeating steps (d)-(f) until completion of the suturing process.
